(19) United States
(12) Patent Application Publication
(10) Pub. No.: US 2021/0283255 A1
(43) Pub. Date: Sep. 16, 2021

(54) Reducing Damage From Chemotherapy And Increasing Cancer Kill Rates By Using Interweaved Low Dose Radiation

(71) Applicant: Radiation Barrier LLC, Henderson, NV (US)

(72) Inventor: Richard Blankenbecler, Henderson, NV (US)

(21) Appl. No.: 16/474,914

(22) PCT Filed: Jan. 2, 2018
(86) PCT No.: PCT/US2018/012106

§ 371 Date: Jun. 28, 2019

Related U.S. Application Data
(60) Provisional application No. 62/441,270, filed on Dec. 31, 2016.

(51) Int. Cl. A61K 41/00

(52) U.S. Cl. CPC A61K 41/0023

(57) ABSTRACT

The present invention provides a method of preventing damage to non-neoplastic cells i.e. healthy cells by irradiating with a low-dose radiation to the non-neoplastic cells, wherein the low-dose radiation is used to initiate a protective cellular response which prevents later damage to non-neoplastic cells by cytotoxic chemical agents or chemo agents and initiating an immune response against neoplastic cells. The low-dose radiation is applied to the sensitive and the non-cancerous organs/cells at a given time before a high dose chemo/drug infusion session.

### CROSS-REFERENCE TO RELATED APPLICATIONS

The present application claims priority to U.S. Provisional Patent Application No. 62/441,270, filed 31 Dec. 2016, the contents of which are considered part of the disclosure of this application and are hereby incorporated by reference in their entireties.

### FIELD OF THE INVENTION

The present invention relates to a method of using radiation therapy in chemo-therapy procedures, more particularly, to the use of interweaving radiotherapy and chemotherapy procedures for the treatment of neoplastic diseases, uncontrolled cell growth, and cancer.

### BACKGROUND

Neoplastic diseases, or cancers, develop when cells do not respond normally to growth regulation signals. Consequently, some or all of their descendants may proliferate inappropriately to produce tumors. Neoplasms that invade surrounding tissues and ultimately spread throughout the body are called malignant neoplasms or cancers. Several ways of treating neoplastic diseases have been developed over the years. The two main methods are radiation therapy and chemotherapy, and new methods are being developed such as immunotherapy.

The present chemotherapy and chemical protocols for the treatment of neoplastic diseases, uncontrolled cell growth, and cancer utilizes various anti-cancer drugs, which are prescribed to the patient. However, these drugs cause inevitable damage to the surrounding healthy cells and to cells far removed from the tumor. These anti-cancer drugs may also cause damage to tissues and sensitive organs which may include but are not limited to heart, intestine, bone marrow, hair follicles, kidneys, resulting in possible follow-on cancers, and shortens lifespan. These side effects can cause considerable discomfort to the patient, thereby increasing the time required for the patient to fully recover from the therapy. Fear of this significant damage, side effects and discomfort can even cause patients to delay or refuse treatment, or cause them to abandon the treatment before it is finished.

To reduce damage to the body, more particularly the surrounding healthy cells and sensitive tissue and organs, extensive searches for chemotherapy and chemical agents that produce minimum damage are required. However, extensive testing to prove the safety of such chemotherapy and chemical agents is a long term and a costly process.

New methods to minimize harmful effects of radiation on healthy cells during cancer treatment have been an intensive research topic. Researchers have found that a low dose exposure of radiation to healthy cells surrounding the cancerous cells induce adaptive response in the healthy cells, which protects the healthy cells from subsequent exposure to harsh chemicals during chemotherapy. A study showing the response of a cell exposure to low dose radiation and high dose radiation was carried out in “Comparison of low and high dose ionizing radiation using topological analysis of gene co-expression networks,” BMC Genomics (2012) by Ray, et al. The experiment was conducted by exposing two different identical cells with a low dose radiation and a high dose radiation and the cells were observed at four time points after exposure to measure the changes in modulation of different gene sets compared to a control sample. It was found that the exposure of cells with low dose radiation resulted in modulation of genes responsible for immune response at three hours after exposure. At 8 hours post exposure, the gene set for metabolic processes and DNA damage, such as regulation of Gl/S stage were expressed. At 24 hours post exposure, changes in gene sets responsible for WNT signaling, Mitotic phase checkpoints, NeK regulation in the cell cycle were observed. It was observed that exposure of a cell to a low dose of radiation modulates one or more genetic pathways responsible for cell repair proteins and immune response for a period of time post exposure.

Researchers have also examined the effect of low dose exposure in order to reduce the harmful effects of high dose exposure in human cells. In this regard, see the review “Global Gene Expression Alterations as a Crucial Constituent of Human Cell Response to Low Doses of Ionizing Radiation Exposure”, National Institutes of Health (2015) by Mykyta Sokolov and Ronald Neumann, hereinafter referred as Sokolov et al. discloses the modulation of cell repair genes in response to low dose exposure. Sokolov et al. discloses that low doses of ionizing radiation changes the gene expression in order to protect the human cells/tissues from harmful effects of challenging dose exposure. After getting irradiated by a low-dose of radiation, the cell initiates a repair sequence and many genes were modulated in the procedure. Afterwards, the genes that produce repair proteins were turned on; the relevant proteins were then produced for a period of time, known to be up to several days. For example, base excision repair (BER) genes and proteins in human BER pathway repairs radiation-induced single-strand breaks, base damage, and basic sites in both nuclear and mitochondrial DNA whereas non-homologous end joining (NHEJ) is involved in fixing DNA double stranded breaks (DSBs) in human cells. In a specific experiment, the peripheral blood mononuclear cells were purified and exposed to priming low dose radiation of 0.1 Gy. After 4 hours, the peripheral blood mononuclear cells were exposed to high dose radiation of 2.0 Gy. The corresponding expression profiles of aforementioned genes and proteins were examined for 30 minutes to 4 hours after the high dose. As a result of low and high dose radiation, the BER genes like APE1, FEN1, LIG1, MBD4 and OGG1 showed up-regulation at mRNA and protein levels in the primed cell. Similarly, NHEJ genes like XRCCS, XRCC6, NHEJ1 and LIG4 were overexpressed at four hours post-irradiation both at the transcript and protein levels. Such kind of overexpression in some BER and NHEJ genes and proteins underlies the active involvement of both BER and NHEJ pathways in human Radio Adaptive Response (RAR). During the procedure followed by other doses, the low dose radiation exposure evoked cellular alert responses to protect against subsequent high dose radiation damage, wherein RAR provided the cellular repair processes.

Similarly, gene expression profiles of DNA Damage Responsive (DDR) genes after low dose radiation exposure and high dose radiation exposure were studied at 1 and 5 hours post irradiation. The level of expression of ATM, ATR, GADD45A, CDKN1A, TP53, CDK2, HDM2, and CCNE was studied using RT-qPCR. The data showed a significant dose-dependent induction of CDKN1A and GADD45A genes upto 1 Gy at 5 hours post irradiation. RAR was observed only with TP53, CDK2, and CCNE.

The aforementioned study and experiment conducted by Sokolov et. al. disclosed that a properly chosen low-dose of radiation applied to a cell, modulates its repair genes. Some were turned on to produce proteins that affect the repair. Other genes were turned off. This latter action can conserve energy needed for the repair, and can also increase the time to the next scheduled mitosis (cell division). This gives more time to affect repairs before the errors can be passed on to the next generation.

Another study in “A History of the United States Department of Energy (DOE) Low Dose Radiation Research Program: 1998-2008” by Dr. Antone L. Brooks, shows that irradiation changes the gene expression in many genes and gene expression was altered as a function of radiation dose, with identified low dose and high dose genes. The aforementioned studies show the modulation of genetic pathway by low dose radiation in the one or more non-neoplastic cell.

Utilizing the above analysis, various methods have been proposed in prior art to generate adaptive response in the healthy cells using low dose radiation before the subsequent exposure to high dose radiation. U.S. Pat. No. 7,963,902 discloses a method that utilizes the adaptive response generated by a low dose radiation in the healthy cells. In this method, the non-neoplastic cells surrounding the cancerous cells are exposed to a low dose radiation that induces metabolic pathways in the healthy cells that increases the probability of survival of the healthy tissues upon various insults such as subsequent radiation therapy. The pre-dose of healthy cells with radiation insures a much higher probability of their long-term survival, and thereby reduces the adverse events associated with radiation therapy. The method disclosed in U.S. Pat. No. 7,963,902 does not utilize other benefits associated with low dose radiation exposure apart from the adaptive response in healthy cells and, therefore, additional improvements in the protocols and extensions to utilize the benefits of low dose radiations are needed. Furthermore, U.S. Pat. No. 7,963,902 focuses on the use of adaptive response to protect the healthy cells from high dose radiation and does not talk about protection of healthy cells against chemo drugs.

Studies have also been conducted to examine other effects of low dose radiation on healthy and cancerous cells. In this regard, studied conducted by Ross in “Consensus of the effect of X-rays on bacterias”, Hygiene Vol. 56, pp 341-344, (1909) first showed that mice treated with low-level radiation were more resistant against bacterial disease. This has been explained by immune response induced by the low dose of radiation. E. J. Broome, D. L. Brown and R. E. J. Mitchel, International Journal of Radiation Biology. 75, 681-690 (1999), have found that low doses of in-vivo beta radiation of mouse skin 24 hr prior to the application of a DNA damaging carcinogen reduced tumor frequency by approximately 5 fold. The low radiation dose activates the repair of DNA breaks. This group has also shown that an adaptive response to low doses of low LET radiation occurs in all organisms thus far examined, from single cell lower eukaryotes to mammals. These responses reduce the deleterious consequences of DNA damaging events, including radiation-induced or spontaneous cancer and non-cancer diseases in mice.

The immune response can be used as an effective weapon against cancer. To do so they need to leave the bloodstream and reach the tumor, but changes in its surroundings often prevent them from doing so. In the study conducted at the German Cancer Research Center (Deutsches Krebsforschungszentrum, DKFZ), “Radiation therapy mobilizes the immune system against tumors”, by Kas/Sel, it is discovered that local applications of low doses of radiation help immune cells escape blood vessels and enter tumor tissue in all mammals tested. However, there is no therapy heretobefore known which provides for the creation of an adaptive response in healthy tissue that is antagonistic to cancer cells in said healthy tissue or nearby cancer tissue.

Thus, there is a need for an approach that utilizes adaptive response in the surrounding healthy cells and sensitive organs and immune response of the body to treat cancer cells. In order to solve the aforesaid problems, the present invention provides a method that not only elicits repair mechanism in healthy tissue but also generates an immune response in both cancerous and nearby cancerous tissues prior to chemo drug infusion to reduce the inadvertent damage to the healthy cells and the sensitive organs, as well as to increase the cancer cell kill rate by applying an interweaving low dose of radiation to non-neoplastic and neoplastic cells in conjunction with chemotherapeutic therapy.

### SUMMARY OF THE INVENTION

The prior art teaches against applying low dose radiation to cancer cells. The present invention advances prior art by eliciting an immediate immune response in the neoplastic cells that outweighs the cellular repair response in neoplastic cells. The low dose radiation neoplastic cellular response is counter-intuitive to prior art. Counter to all previous studies, low dose radiation on the cancer cells has been shown to increase cancer kill rates to as high as 5 fold.

The present invention addresses the needs in the art by providing a method for protecting normal healthy cells and sensitive organs from chemotherapy and chemical agents and eliciting an immune response against cancerous mass. Once so protected, a patient may receive chemotherapy and experience a reduction or elimination of adverse events such as damage to organs and tissues, follow-on cancers, shortened lifespan and considerable patient discomfort.

In a first aspect of the present invention, a method of killing cancerous cells comprising: a) administering a low dose radiation to neoplastic tissues and non-neoplastic cells surrounding neoplastic tissues and non-neoplastic cells sensitive to a chemo-drug; wherein said low dose radiation elicits antibodies against neoplastic tissues and elicits a repair mechanism in the non-neoplastic cells and in the non-neoplastic cells sensitive to the chemo-drug; and wherein said low dose radiation on neoplastic tissues causes anchors to form in the blood vessels within said neoplastic tissues that aids in latching of antibodies to anchors, allowing the antibodies to enter nearby neoplastic cells and kill them; b) waiting for a period of 48 to 72 hours and infusing a chemotherapeutic drug to act upon said neoplastic tissues. Irradiating the non-neoplastic cells modulates one or more genetic pathway responsible for cell repair protein and thus, helps in generating protective adaptive response in the non-neoplastic cells. The modulation of genetic pathway by low dose radiation on the non-neoplastic cell can be used to determine the reaction of one or more pharmaceuticals or chemical agents on said one or more non-neoplastic cell. Additionally, the modulation of genetic pathway by low dose radiation on the non-neoplastic cell can be used to protect against radiation hazards (e.g. radiation workers, first responders, and astronauts). The low dose radiation applied on sensitive tissues and non-neoplastic cells is in the range of 5 cGy to 20 cGy. The non-neoplastic cells are in contact with or in close proximity to a target neoplastic cell of a neoplastic disease or may be distantly located (organs sensitive to chemo drug). The method can be used as a method for therapeutic treatment of cancer with chemotherapy. The low dose can be administered by a neutron beam as well as by a standard x-ray/gamma beam.

In a second aspect of the present invention, a method for killing cancerous cell comprising: a) targeting a tumor tissue, tissues sensitive to a chemo drug and one or more non-neoplastic cells present in vicinity of a tumor tissue with a predetermined low dose of radiation, wherein said low dose radiation induces an immune response against tumor tissues, and a cellular repair process in said one or more non-neoplastic cells and tissues sensitive to the chemo drug; wherein the low dose radiation on the tumor tissues causes anchors to form in the blood vessels within said tumor tissue that aids in latching of antibodies to anchors, allowing the antibodies to enter nearby tumor cells and kill them; waiting for 48 to 72 hours and infusing the chemo drug to act upon the tumor tissue; and wherein the above steps are repeated till the recommended dose of high radiation is completed. The low dose radiation modulates one or more genetic pathway of non-neoplastic cells to induce cell repair proteins. The applied low dose radiation is in the range of 5 cGy to 20 cGY. The immune response initiated by the low dose radiation inhibits the proliferation of neoplastic cells. The immune response protects the healthy non-neoplastic cells and tissues sensitive to the chemo drug from the chemo drug.

In a third aspect of the present invention, a method for killing cancerous cells is provided. The method comprises: a) administering a low dose radiation to neoplastic tissues, non-neoplastic cells surrounding neoplastic tissues and non-neoplastic cells sensitive to a chemo-drug; wherein said low dose radiation elicits antibodies against neoplastic tissues and elicits a repair mechanism in the non-neoplastic cells and in the non-neoplastic cells sensitive to the chemo-drug; and wherein said low dose radiation on neoplastic tissues causes anchors to form in the blood vessels within said neoplastic tissues that aids in latching of antibodies to anchors, allowing the antibodies to enter nearby neoplastic cells and kill them; waiting for a period of 48 to 72 hours and administering a second predetermined low dose radiation to the non-neoplastic cells surrounding neoplastic tissues and non-neoplastic cells sensitive to the chemo-drug, wherein said second predetermined low dose radiation elicit repair mechanism in said non-neoplastic cells; waiting for 24 hours and infusing a chemotherapeutic drug to act upon said neoplastic tissues. The low dose radiation applied is in the range of 5 cGy to 15 cGy, which modulates the genes responsible for repair mechanism in the non-neoplastic cells. The antibodies against neoplastic tissues prevent invading neoplastic cells from entering into non-neoplastic cells. The repair mechanism in the non-neoplastic cells protects the cells from the chemo drugs.

It is possible to combine the new protocol that utilizes the immune response with the prior art as described in U.S. Pat. No. 7,963,902 (the content of which is incorporated herein by reference in its entirety) to achieve a more effective treatment. Three example protocols that make use of the repair and immune response are the following: In the prior art, the proposed protocol was a low dose irradiation of the surrounding healthy cells followed by about 24 hours later with the standard chemotherapy applied to the cancer cells which may be repeated over several days. This protocol is changed by applying a low dose to both the healthy and cancer cells and then 24-48 hours later applying the standard chemo treatment. A third protocol is to apply a low dose to both the healthy and cancer cells, wait for around 24-48 hours, apply the low dose to the healthy cells and then after 24 hours start the high dose standard chemo treatment. Other similar sequences are clearly possible.

### BRIEF DESCRIPTION OF THE DRAWINGS

FIG. 1 is a table of the effects of radiation dosages in accordance with an embodiment of the present invention.

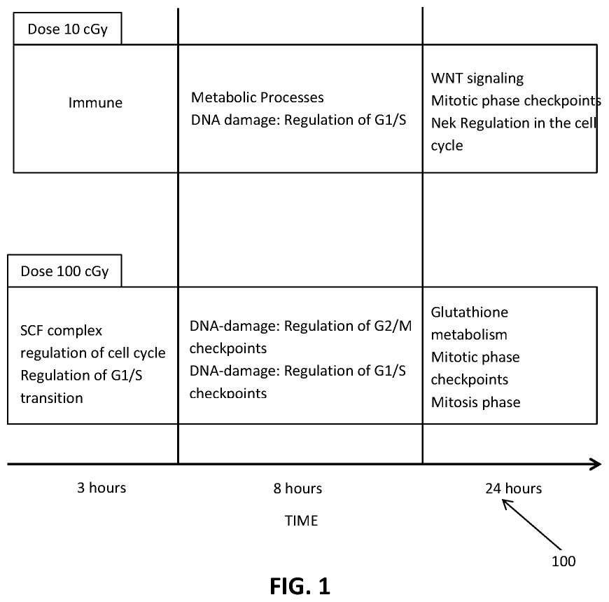

### DETAILED DESCRIPTION OF THE INVENTION

In the following detailed description of embodiments of the invention, numerous specific details are set forth in order to provide a thorough understanding of the embodiments of the invention. However, it will be obvious to a person skilled in the art that the embodiments of the invention may be practiced with or without these specific details. In other instances, well known methods, procedures and components have not been described in details so as not to unnecessarily obscure aspects of the embodiments of the invention.

Furthermore, it will be clear that the invention is not limited to these embodiments only. Numerous modifications, changes, variations, substitutions and equivalents will be apparent to those skilled in the art, without parting from the spirit and scope of the invention.

As used herein, the terms neoplastic, cancer, and tumor are used interchangeably to indicate a cell, tissue, or condition in which there is uncontrolled or abnormally fast growth of one or more cells of a particular type. The invention is applicable to neoplastic cells of cancer or tumor in all of its forms. Such growth can happen in vivo to produce a mass of cells within an organism, such as a human, or can occur in vitro to produce a culture of cells that might or might not have characteristics of cell lines. Accordingly, such cells or tissues can be, but are not necessarily, immortal such as stem cells. Likewise, the cells or tissues can be, but are not necessarily, primary cells obtained directly from a cancerous or a non-cancerous tissue.

As used herein, the term sensitive cells, sensitive tissues, and sensitive organs imply the non-neoplastic cells or tissues or organs on which a particular chemo drug or agent has an adverse effect, thus hampering their normal activity.

Furthermore, as used herein, the terms radiation (and all of its forms) and electromagnetic energy are used interchangeably to indicate energy of one or more wavelengths of the electromagnetic spectrum. The invention is not limited to the use of a particular wavelength, but instead can be used with any wavelength of the electromagnetic spectrum. For example, the invention contemplates use of a particular wavelength of energy that can activate a substance that can absorb one wavelength of energy and re-emit at another wavelength. For ease of reference, electromagnetic energy is typically referred to herein as radiation, and this term is to be broadly interpreted.

More specifically, radiation is energy that comes from a source and travels through some material or through space. Thus, light, heat, and sound are types of radiation. One useful type of radiation according to the present invention is ionizing radiation, which is radiation that can produce charged particles (i.e., ions) in matter. Ionizing radiation is often produced in the medical setting by man-made devices, such as CT-Scan, X-ray, or Linear Accelerator Machines (LINAC). It is well known that ionizing radiation can be produced by unstable atoms (i.e., radioactive atoms), which are atoms that have an excess of energy, mass, or both, and which shed or emit that energy and/or mass in the form of radiations in order to achieve a stable state. For the purposes of this invention, it is to be understood that there are two kinds of radiation: electromagnetic (e.g., light, gamma radiation, X-rays, ultraviolet) and particulate (e.g., proton or neutron emission, beta and alpha radiation).

Furthermore, as used herein, the terms chemotherapy agent (and all of its forms), chemo agent, chemo drug and chemical agents are used interchangeably.

It is also to be understood that, where the invention relates to therapeutic treatment of a subject, a diagnosis of a localized cancer has been made and the size, shape, and location of the cancerous mass has been determined by standard methods known in the art. In other words, it is to be understood that the invention relates to in vivo of a patient in need thereof, and the routine procedures for identifying such patients and characterizing their tumor(s) have been performed. By subject, it is meant any living organism in which a neoplasia may exist. Thus, a subject may be, but is not limited to, a human or other animal (e.g., a dog, cat, horse, bird, or other companion or agricultural animal). As used herein, the terms subject, patient, person, and animal, unless otherwise indicated, are used interchangeably to indicate a living organism in which a neoplasm may exist. Accordingly, the present invention has application in both the human health field and in veterinary medicine.

The present invention discloses a method for protecting normal healthy cells and sensitive organs from chemotherapy and chemical agents. The method provides prevention from damage to non-neoplastic i.e. healthy cells, which occurs during standard chemotherapy sessions. For this purpose, low-dose radiation is used to initiate a protective cellular response which prevents or reduces damage to non-neoplastic cells by cytotoxic chemical agents. After pre-dosing a healthy cell with a low dose radiation, the cell responds by modulating genes that produce repair proteins that control certain cell functions, which include creating immune response among others. These proteins then proceed to repair the damage to the cell, a process that lasts for days. The present invention also discloses a method of preventing damage to non-neoplastic cells, wherein the low-dose radiation is applied to sensitive tissues and organs before the chemo drug infusion session.

In an embodiment, the present invention also provides a method for eliciting an immune response against cancerous cells. When a low dose radiation is applied to healthy cells and neoplastic cells, it generates an immune response in the body against cancerous cells by generating antibodies against them. When the neoplastic or tumor cells are exposed to low dose radiation, it helps them to form anchors on the blood vessels, the antibodies latches on to these anchors, exit from blood vessels and kill the invading cancerous cells that tried to escape into the healthy cells.

The invention relates to in vivo and in vitro treatment of cells. In aspects relating to in vivo uses, it is generally a method of therapeutic treatment, which can be curative or prophylactic. Thus, the method can be practiced on a subject suffering from a neoplastic disease, such as the one in which a neoplastic mass is growing, to reduce the growth of, reduce the size of, or eliminate the neoplastic mass. In addition, the method can be practiced on a subject who previously suffered from a neoplastic disease, such as the one who had a neoplastic mass removed by surgery or radiation treatment, to ensure that all neoplastic cells of the mass are killed. The present invention provides particular protocols for pre-dosing healthy cells and tissues, especially those that are very sensitive to the chemotherapy substance, with low-dose radiation, while avoiding irradiating cancerous cells, in order to induce a cellular repair response in the healthy cells/tissues. This is then followed by any standard chemotherapy protocol therapy.

As a general matter, the method described herein relates to pre-treating healthy tissues that are at risk from standard chemotherapy agents, including those surrounding the neoplastic growth and other organ tissues sensitive to the chemotherapy agents, with a low-dose radiation. This low dose radiation exposure results in an adaptive response in the irradiated cells and tissues that increases the probability of survival of healthy tissue upon various insults such as those arising from subsequent cancer therapy. The neoplastic tissues are also exposed to low dose radiation, which initiates an immune response against the cancerous cells by causing anchors to form in the blood vessels within said cancerous cells. The antibodies latches to the anchors and thus allows the antibodies to enter nearby cancerous cells and kill them. The immune response starts as early as 1 hour of exposure to low dose radiation and remains active for few days up to 72 hours.

During a subsequent chemotherapy protocol designed to kill the cancerous cells, the healthy cells and sensitive organs which may include but are not limited to heart, intestine, bone marrow, hair follicles, kidneys and other chemo-agent sensitive tissues in the body will inevitably be damaged as well. Also, tissues that are not in the vicinity of the cancerous tissue but are very sensitive to the chemotherapy agent can also be damaged. The pre-dose of radiation to the healthy cells and sensitive organs and tissues insures a much higher probability of their long term survival, and thereby reduces these adverse events.

For the low-dose radiation applied in the range of 0.05 Gy to 0.15 Gy, a cell initiates a repair sequence where many genes are modulated. The genes that produce repair proteins are turned on; the relevant proteins are then produced for a period of time. The production is known to start after about 6 hours and lasts upto few days. As these proteins are produced and move throughout the cell, they start repairing damage. Since this active repair period lasts for days, if the cell is then damaged again during this time, for example by a standard chemotherapy treatment, the repair commences immediately and at near full strength.

If a properly chosen low-dose of radiation, under aspects of the present invention, has been applied to a cell, its repair genes are modulated. Some are turned on to produce proteins that affect the repair. Other genes are turned off. This latter action can conserve energy needed for the repair and can also increase the time to the next scheduled mitosis (cell division). This gives more time to affect repairs before the errors can be passed on to the next generation.

Standard chemotherapy protocol requires administration of a selected cytotoxic chemical agent or chemo agent into the patient in a number of separated sessions, perhaps a few days apart. Under aspects of the present invention, each of these sessions involves exposure of a low dose radiation in the range of 0.05 to 0.15 Gy to the healthy sensitive cells and tissues followed by the chemo infusion process after 48 to 72 hours of the low dose radiation exposure to sensitive cells and tissues. During this time period, immune response induced in the patient body acts on the cancerous cells and also prevents establishment of neoplastic cells in adjacent healthy tissues. The time spacing between the radiotherapy exposure and the chemo-agent infusion is chosen to maximize the efficacy of the adaptive response in repairing damage suffered by healthy cells.

The invention provides particular protocols for pre-dosing healthy cells and tissues with low-dose radiation, while avoiding irradiating cancerous cells, in order to induce a cellular repair response in the healthy cells/tissues, followed by a chemotherapy protocol. In embodiments, it has been found that a pre-dose as small as 1 mGy (in Gray Units) can induce a protective cellular adaptive response. For better understanding of the physical mechanisms arising from a radiation dose, note that a dose of one mGy corresponds roughly to one radiation track per cell. The average radiation exposure from natural sources is 3 mGy/yr and from human activity 1 mGy/yr. The total radiation dose from a chest CT scan or an abdominal CT scan is approximately ˜110 mGy=1 cGy.

In embodiments, the strategy is to select and use those chemo agents which can be effectively stopped by the adaptive repair processes triggered by a pre-dose of low-dose radiation. Such selection offers the optimum protection for the healthy cells after exposure to a pre-dose of low-dose radiation. After pre-dosing a healthy cell with low-dose radiation, the cell responds by modulating genes that produce repair proteins and that control certain cell functions, and initiate immune response. These proteins then proceed to repair the damage to the cell, a process that lasts for several days. Certain processes that are a drain on the cell are slowed down so that the repair can proceed as rapidly as possible, optimally completed before the next cell division. This slowing of cellular reproduction is also a benefit of the pre-dosing regimen because chemotherapeutic agents affect rapidly dividing cells.

In chemotherapy, a chemical agent is chosen that attacks, damages, and eventually kills rapidly reproducing cells. The cell lifecycle is conventionally divided into 5 phases. Each chemotherapeutic agent will attack a rapidly growing cell during one of the phases. In accordance with the embodiments, chemotherapy is normally applied in defined time cycles, for example, once a week or once every three weeks, as prescribed by the oncologist. Furthermore, the day of chemotherapy application is often preceded one day earlier by a pre-treatment medication and the day after by a blood count booster treatment. These are used to help the patient recover from the side effects of the therapy. To restate the main idea of this therapy, the targeted low-dose radiation therapy offers protection to the healthy cells of the body while denying that same protection to cancerous cells. The immune response acts on the cancerous cells and also prevents the escape of neoplastic cells to adjoining healthy tissues.

In a situation where a localized tumor is in the interior of the body, within or at the surface of an organ, a low radiation dose is applied to the cells surrounding the tumor in the organ prior to the injection of the chemotherapy agent. If there are remote organs or cells that are particularly sensitive to the specific chemo-agent used, then they are also provided with low-dose protective irradiation. The adaptive response of the cell to a low-dose of radiation is in the range of 0.01-0.4 Gy that causes the modulation of many genes, including those that are involved in DNA-RNA repair. Other modulated repair genes include cell-cycle control, heat shock, ion regulation and membrane repair. Still others involve myelin and protein synthesis repair and an immune system response. These up-regulated genes produce their repair proteins over a period of hours and these proteins persist for at least two days. Once the cells are protected, and prior to the expiration of the repair proteins, most preferably when repair protein levels are at peak amounts, or preferably when they have not degraded to baseline levels present at pre-radiation time, a selected chemotherapy agent is injected whose main modes of cell killing can be counteracted by the repair gene proteins produced by the adaptive response. The cancer cells, that did not receive the low radiation dose and are hence unprotected, are killed by the chemo agent as usual, whereas the low radiation dosed cells are able to recover from the chemotherapeutic damage, most preferably prior to cell division.

Furthermore, many cell types in the body are very sensitive to damage at sufficiently high doses of chemotherapeutics. Among these are bone marrow, mucosal cells of the intestinal tract, liver and kidney cells, epithelial cells and nervous system cells. It is thus possible to protect these cells by applying a low-dose radiation to turn on their repair mechanisms and these cells are especially suited to usage of the protocols specified herein, e.g. a low pre-dose of radiation in the range of 0.01-0.4 Gy.

As discussed above, preferred chemotherapeutic agents are those which affect cell cycle stages that are susceptible to the cellular repair proteins produced after an exposure to low level of radiation and may be selected from the classes of alkylating agents, anti-metabolite agents, and topoisomerase agents: The alkylating class of chemotherapeutic agents (originally derived from mustard gas) bind covalently to the DNA, RNA and to some protein molecules. This prevents normal mitosis, thereby inducing apoptosis (death) of the cell. The repair proteins present in the cell after the low-dose treatment work to return the affected (alkylated) DNA, RNA and protein molecules to their normal state. The anti-metabolite class of chemotherapeutic agents operates by impeding the synthesis of DNA and RNA by incorporating themselves into their structure. The repair proteins produced by low-dose radiation restore the DNA and the RNA to their normal state. The topoisomerase class of inhibitors operates by not allowing the DNA to stretch and unwind during replication or transcription. The repair proteins produced by low-dose radiation restore the equilibrium situation of the DNA, allowing normal replication of the cells.

In embodiments, a protocol is used that does not disrupt the normal chemotherapeutic schedule. For example, the day of chemo application is often preceded a few days earlier by a pre-treatment and the day after by another booster treatment. These are used to help the patient recover from the side effects of the therapy. Therefore, the day before the chemo application could also be used for a low-dose radiation session.

In embodiments one or more chemotherapeutic agents may be used in combination or series according to their standard protocols of prescription, with low-dose irradiation as described herein being applied to the non-cancerous tissues. In embodiments the noncancerous tissues may be selected from those most susceptible to the selected chemotherapeutic(s) or those tissues that are most likely to be the targets of migrating cancer cells. In other embodiments, the low-dose irradiation as described herein may be applied to the whole body, excepting the tumor tissue. In further embodiments, non-neoplastic cells which would suffer non-killing damage are protected from such damage by the above methods.

The low-dose radiation is applied to the healthy cells present in the patient's body that are at risk from the chemotherapy agent, as the chemotherapy treatment is systemic. Then after a selected wait period, a standard chemotherapy treatment is applied. The low-dose radiation may be applied prior to each session of a chemotherapy treatment regimen. Since the healthy cells inevitably receive a toxic chemical exposure during chemotherapy sessions, they now have the extra protection of the adaptive response initiated by the low-dose radiation applied prior to the chemotherapy. The beneficial effect of low dose radiation on the healthy cells for inducing protecting response in them is studied in the research article, entitled “Beneficial effects of low dose radiation in response to the oncogenic KRAS induced cellular transformation” by Kim et al.

In another embodiment of the present invention, a method to kill cancer cell is provided. The method comprising administering low dose radiation to neoplastic tissues, non-neoplastic cells surrounding neoplastic tissues and non-neoplastic cells sensitive to a chemo-drug; wherein said low dose radiation elicits antibodies against neoplastic tissues and elicits a repair mechanism in the non-neoplastic cells and in the non-neoplastic cells sensitive to the chemo-drug; and wherein said low dose radiation on neoplastic tissues causes anchors to form in the blood vessels within said neoplastic tissues that aids in latching of antibodies to anchors, allowing the antibodies to enter nearby neoplastic cells and kill them; (b) waiting for a period of 48 to 72 hours and infusing a chemotherapeutic drug to act upon said neoplastic tissues. The administration of low dose radiation to neoplastic tissues elicits an immune response in the neoplastic tissues which helps in killing the cancerous cells.

### Example 1

As an example, in our most recent experimentation, human subjects with epithelial skin cells were treated in-vivo with two methodologies. The first patient received an interweaving low dose radiation of 10 cGY, specifically to the healthy tissue surrounding the localized skin cancer. The second patient also received low dose radiation of 10 cGy to both the neoplastic cells and healthy cells adjacent to the tumor. Both patients underwent biopsies before treatment, 24 hours after the low dose treatment, and then one week later after standard therapy.

The protocols were tested in-vivo, with DNA analysis verifying the effect of low dose radiation: a) the excitement of a cellular repair adaptive and immune response in healthy tissue surrounding the neoplastic cells. b) the excitement of a cellular repair adaptive response in neoplastic cells that is outweighed by the immune response in the neoplastic cells that increases cancer kill rates by upto 5 fold. To clarify, the low dose radiation elicits antibodies against neoplastic tissues and elicits repair mechanisms in non-neoplastic cells and cells sensitive to the chemo-drug; and wherein said low dose radiation on neoplastic tissues causes anchors to form on said neoplastic tissues that aids in latching of antibodies onto said neoplastic tissues.

Schedules for Radiation-Chemo Sessions

To induce protective adaptive response in non-cancerous sensitive tissue and organs, and to utilize the protective adaptive response for scheduling chemotherapy session for a patient, the present method targets the organs that are sensitive to a particular chemo-agent with a predetermined low dose radiation. The method may also involve targeting the non-neoplastic cells that may or may not be in contact with or in close proximity to the target neoplastic cell with the predetermined low dose radiation. The low dose radiation induces a cellular repair process in the targeted chemo-agent sensitive organs and the non-neoplastic cell. Many genes are modulated. Each of the modulated repair genes can initiate a complicated pathway that involves the excitation of many other subsequent genes. The study of these pathways serves an important role in the development of chemotherapy and other drug agents for treating tumorous and cancerous tissues. The use of low dose radiation to excite these pathways can have many advantages over chemical excitation.

In an embodiment of the present invention, a method for treating at least one neoplastic cell with a harmful amount of electromagnetic energy is provided. The method involves the induction of adaptive response in healthy cells surrounding the tumor mass so that they may be able to withstand the harmful effect of subsequent high dose exposure of radiation. The method also involves initiating an immune response in the body. It has been found during the experiment that a low dose exposure to cancerous cells and healthy cells initiate an immune response in the body against cancerous cells.

The method for treating at least one neoplastic cell comprises: a) administering a low dose radiation to neoplastic tissues and non-neoplastic cells surrounding neoplastic cells within 0.1 to 3.0 cm of a tumor; wherein said low dose radiation elicits repair mechanism in the non-neoplastic cells and antibodies against neoplastic tissues; and wherein said low dose radiation on neoplastic tissues causes anchors to form on said neoplastic tissues that aids in latching of antibodies on to said neoplastic tissues; (b) waiting for a period of 48 to 72 hours and administering a high dose radiation to neoplastic tissues. During this time period, immune response induced in the patient body acts on the cancerous cells and also prevents establishment of neoplastic cells in adjacent healthy tissues. The time spacing between the radiotherapy exposure and the high dose radiation application is chosen to maximize the efficacy of the adaptive response in repairing damage suffered by healthy cells.

The low dose radiation on the healthy cells modulates repair protein genes in the cell to induce protective adaptive response in non-cancerous tissue. The method utilizes this protective adaptive response of cell for modifying the radiation therapy dosage schedule. The pre-dose of the healthy cells with low dose radiation insures a much higher probability of their long term survival, and thereby reduces the adverse events associated with radiation therapy. The effect of radiation on a cell depends on the type of cell and the amount of radiation and its dose rate. For example, a muscle cell, a liver cell, and a breast cell, etc., react to radiation in different ways and the scale of the reaction depends on the radiation beam parameters. In choosing the specifics of the optimum low-dose radiation beam for treatment, these radiation beam parameters are chosen corresponding to the cell type being irradiated.

After pre-dosing a healthy cell with a low dose radiation, the cell responds by modulating genes that produce repair proteins that control certain cell functions, which include creating immune response among others. These proteins then proceed to repair the damage to the cell, a process that lasts for days. The low dose radiation induces metabolic changes in the non-neoplastic cells and modulates the repair protein genes responsible for cell repair mechanism. As these proteins are produced and moved throughout the cell, they start repairing the damage. The protein producing genes remain activated for a period of time, up to several days; therefore the relevant protein is being produced and keeps on repairing the damaged cells for that time period. Since, the active repair period lasts for days, if the cell is then damaged again during this time, for example by standard high-dose radiotherapy, the repair commences immediately and at near full strength.

Exposure with low dose radiation also turns off other genes; this action conserves energy needed for the repair and also increases the time for the next scheduled mitosis (cell division). This gives more time to affect repairs before the errors can be passed on to the next generation.

Table 1 represents some of the genes that are known to respond to the low dose radiation:

TABLE 1

| | Number | Responsive | |
|---|---|---|---|
| Group | of Genes | Genes | Function |
| I | 11 | MBD4, OGG1 | Base excision repair (BER) |
| II | 6 | APEX1, LIG3, | Other BER and strand break |
| | | PNKP | joining factors |
| III | 3 | PARP1, PARP2 | Poly(ADP-ribose) polymerase |
| | | | (PARP) enzymes |
| IV | 3 | MGMT | Direct reversal of damage |
| V | 2 | TDP1 | Repair of DNA-topoisomerase |
| | | | crosslinks |
| VI | 10 | MSH2 | Mismatch excision repair (MMR) |
| VII | 24 | XPC, DDB2, | Nucleotide excision repair (NER) |
| | | LIG1 | |

The exposure of low dose radiation on neoplastic cells or tumor cells have effects, such as initiation of immune response in the body. The immune system usually recognize cancer cells and “killer T cells” invade the tumor tissues. Normally immune cells migrate into tissues through “anchors” formed by blood vessels. As the invading immune cells flow through blood stream, they latch onto the anchors and can thus leave the bloodstream. The problem with tumors is that they often prevent the anchors from forming, which prevents the killer T cells from using these exit points. The exposure of cancerous cell to low dose radiation leads to the formation of anchor molecules in the vessel wall. Additionally, the low dose exposure to healthy cells and cancerous cells results in generation of antibodies against tumor cells that lasts for several days. A low dose radiation, striking as far away as 1.5 cm from the surface of a tumor, will excite an immune response not only in healthy tissues but also in nearby tumor cells. Due to the excitation of immune response, the growth of these tumor cells is then reduced. The immune response generated by the low dose radiation has an additional benefit, in that it prevents the cells from the tumor mass to invade the surrounding healthy tissues.

The main pathways of the immune response triggered by the low dose radiation includes, but are not limited to: a) Altered T cells and B Cell Signaling; b) Antigen presentation pathway; c) B cell development; d) OX40 Signaling Pathway. These pathways result in the production of molecules associated with Dendritic cell maturation, NF-kB signaling, and Fcy receptor-mediated Phagocytosis in macrophages and monocytes.

It was found that this immune response is active within 24 hours of the exposure, whereas a natural trigger normally requires 6-8 days to become fully active. This quick response could be a very important feature in the application of low dose radiation. The low dose turns on the natural immune response as well as the induced adaptive response.

Another effect of low dose radiation is the activation of immune response, which acts on the cancerous tissue and also prevents escape of neoplastic cells to the surrounding healthy tissues. After waiting for 48 to 72 hours, the chemotherapy agent can then be administered to the patient.

In an embodiment of the present invention, the low dose radiation is in the range of 5 cGy to 15 cGy, wherein the low dose may be administered by a neutron beam or as well by a standard x or gamma-ray beam. In an embodiment of the present invention, the time period between the low dose radiation and administration of a chemotherapy dose, which can be termed as the wait time is between 6 and 48 hours.

Chemotherapy drugs are administered in many ways: orally, injection into different regions of the body, implantable wafers, and topically in the protective time window.

A low dose radiation will cause an adaptive response that turns on certain repair genes. These genes then produce a set of proteins that proceed to repair the damages to the cell. The production of these proteins starts a few hours after exposure and then proceeds for few days after irradiation with the low dose radiation. This time period is the protective window offered to irradiate sensitive cells and tissue by the low dose radiations.

Chemotherapy is usually given in cycles with rest periods between two sessions, so as to allow the patient to recover from the effect of the treatment and to allow the cancer or tumor cells to be attacked at their most vulnerable times. Normally a premedication is given to reduce the worst side effects. The infusion sessions can be scheduled for several consecutive days, weekly or even longer periods.

In an embodiment of the present invention, various possible schedules for combination of low dose radiation and chemo sessions are provided that uses the time dependence of the adaptive response generated in the irradiated sensitive cells and tissues (protective window). Practical schedules of the standard chemotherapy sessions are restricted by the standard work week and work rules.

There can be various possible schedules such as:

Once a Week:

In an exemplary embodiment, when the oncologist has determined the chemotherapy session has to be administered to patient is once a week, then the patient is administered with the determined chemo drug in the protective window period. In this case, prior to administration of the chemo drug or agent, the tissue and organs of the patient sensitive to that particular chemo drug or agent and tumor tissue is irradiated with the low dose radiation so as to modulate the protective adaptive response in the sensitive tissues and organs. Then after a wait time of approximately 48 to 72 hours, the patient is administered with the chemo drug or agent. The administration of chemo drug or agent to the patient is done within the protective time window.

Three Consecutive Days:

In another exemplary embodiment, if the chemotherapy session as determined by an oncologist for the patient is three consecutive days, then according to the embodiments of the present invention, the session can be scheduled as follows: firstly exposure of low dose radiation is given to sensitive cells and tissues of the patient and neoplastic tissues, then waiting for 48 to 72 hours, and then performing first dose of chemo drug infusion. Then after a wait period of 24 hours, second dose of chemo drug is administered and then after a wait time of 24 hours, third dose of chemo drug is administered to the patient.

In an embodiment of the present invention, the patient can be irradiated with multiple low dose radiation sessions in a week, depending on the intensity of the chemo treatment.

In another embodiment of the present invention, a method for interweaving one or more low dose radiation session with one or more chemotherapy sessions is provided to prevent the harmful effects of chemo drugs on to healthy sensitive cells of the human body. According to the method one or more non-neoplastic cells or tissues or organs, that may not be in the vicinity of the cancerous cells but are particularly sensitive to the particular chemotherapy agent, and the neoplastic tissues are targeted with a predetermined low dose radiation. The low dose radiation induces a cellular repair process in said one or more non-neoplastic cells or tissues or organs and immune response in neoplastic tissues. The repair process remains active during a protective window period. Then a wait period of 48 to 72 hours is allowed to pass. Following the wait period, varying levels of the chemotherapy agents are administered to the patient till the protective window period is over. If the dose of the chemo agent has not been completed and the protective window period is over, then before administering another dose of chemo agent, the sensitive cells or tissues or organs are again irradiated with a low dose radiation. The next remaining dose of chemotherapy agents or drugs is then administered to the patient in the protective window period of second low dose radiation. This process is repeated till the complete scheduled dose of chemo drug has been administered to the patient.

There are many other possible schedules that interweave the low dose radiation session with the chemo infusion sessions and still make full use of its protective properties.

In this way, the present invention provides a therapeutic treatment of cancer with chemotherapy preceded by a low dose radiation exposure. To clarify, if a low dose radiation is applied to both the tumor and the surrounding healthy cells, then the repair genes in both cell types will be activated. However if the immune response in the cancer cells is stronger than its repair mechanism, then the cancer cells will undergo a net negative effect. Preliminary results on human cells have indicated that the immune response is several times (up to 5 times) stronger than the repair response. Following the low dose exposure, a standard chemotherapy procedure is applied.

In an embodiment of the present invention, the method of using low dose radiation to induce a protective adaptive response in the cells or tissues or organs sensitive to a chemotherapy agents or drugs can be used to increase the number of chemo drugs that can be used in therapy. Different chemo drugs have different efficiencies in killing cancerous or tumor cells. Some are extremely efficient in killing cancerous cells, but due to their lethal effect on healthy cells, the clinical use of these drugs is very restricted. The method of the present invention can be used to devise a strategy for using these chemo drugs. In an embodiment, the method comprises: identifying a chemo drug that is effective against cancerous cells; identifying one or more healthy cells or tissues that are sensitive to the chemo drug; irradiating the one or more sensitive cells or tissues with a low dose radiation; then administering the chemo drug to the patient during the protective window of the low dose radiation to optimize the cancer kill rates while sparing the patient from the lethal effects of the chemo drug on healthy tissue or cells.

Consider a chemo drug that is extremely effective in killing cancerous cells but is also quite lethal to certain healthy cells. If the application of low dose radiation activates repair genes that are able to sufficiently repair damage caused by the chemo agent, then this agent can be used to treat patients following a low dose exposure. For example, cancer chemotherapy drugs such as anthracycline antibiotics are used to treat many types of cancers, but their main adverse effect is cardiotoxicity. This is disclosed in research article “Cardiovascular toxicity induced by chemotherapy, targeted agents and radiotherapy: ESMO Clinical Practice Guidelines. Annuals of Oncology Oct;23 Suppl 7, vii155-66”. For the use of anthracycline antibiotic for treatment of cancer, the present method can be used by exposing the cardiac cells to low dose radiation sufficient to induce an adaptive response. Thereafter, in the protective time window, the anthracycline antibiotics can be used to treat cancer cells without inducing serious cardiotoxicity.

In another embodiment of the present invention, the invention can be used on a patient with a tumor as well as on post-op patients whose tumor has been removed after a surgery. In a patient where a surgical procedure has removed or debulked a tumor, the chemotherapy process is generally employed to kill any tumor debris left over after the procedure. Before administering the chemo drugs, the sensitive cells and tissues are exposed to low dose radiation for inducing the protective adaptive response in these cells.

In another embodiment of the present invention, the method of killing cancerous cells comprises of: (a) administering a low dose radiation to neoplastic tissues and non-neoplastic cells surrounding neoplastic cells and cells sensitive to a chemo-drug; wherein said low dose radiation elicits antibodies against neoplastic tissues and elicits a repair mechanism in the non-neoplastic cells and in non-neoplastic cells sensitive to the chemo-drug; and wherein said low dose radiation on neoplastic tissues causes anchors to form in the blood vessels within said neoplastic tissues that aids in latching of antibodies to anchors, allowing the antibodies to enter nearby neoplastic tissues thus killing them; (b) waiting for a period of 48 to 72 hours and infusing a chemotherapeutic drug to act upon the said neoplastic tissues.

In other embodiments, the application of low dose radiation can also be used to excite an immediate immune response in the body, more quickly and efficiently than the body's typical immune response to cancer cells (24-48 hours vs. approximately six days). The irradiation of healthy body cells induces DNA damage in the cell which alerts the immune system by signals displayed on the cell surface. This effect has a strong link to the innate immune system and tumor surveillance. The activation of the immune response is an important tool in the study of the effects of low dose radiation and its effects on cancer. The immune response will combat local inflammation and also retard the growth of the cancer by attacking cancer cells located near or in the irradiated tissues as well as throughout the body. Due to induction of the immune response by the low dose radiation, an adaptive response throughout the entire body is produced and adverse effect of subsequent treatment with the chemotherapy agent on the healthy cells and tissues is prevented.

In another embodiment, the method of present invention can be used in similar possible sequences. For instance, the method comprises administering low dose radiation to health cells (non-neoplastic cells) that are adjacent to tumor tissue or are distantly located which are sensitive to a particular drug and to the neoplastic tissue. The low dose radiation on healthy cells elicit adaptive protective response and on tumor tissues, it elicits an immune response. Then a wait period of 48 to 72 hours is observed, during which antibodies can act on the tumor tissue. Thereafter, a low dose radiation is again administered to the healthy non-neoplastic cells, so that the adaptive response in them is triggered. After a wait period of 24 hours, chemotherapy agents are infused in the body to act upon remaining cancerous cells. The present invention envision another possible schedules for interweaving low dose radiation on non-neoplastic cells and neoplastic cells with chemo-drug infusion.

It will be apparent to those skilled in the art that other embodiments of the invention will be apparent to those skilled in the art from consideration of the specification and practice of the invention. While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention. It is intended that the specification and examples be considered as exemplary, within the true scope and spirit of the invention being indicated by the claims.

We claim:

1. A method of killing cancerous cells comprising:
(a) administering a low dose radiation to neoplastic tissues, non-neoplastic cells surrounding neoplastic tissues and non-neoplastic cells sensitive to a chemo-drug;
wherein said low dose radiation elicits antibodies against neoplastic tissues and elicits a repair mechanism in the non-neoplastic cells and in the non-neoplastic cells sensitive to the chemo-drug;
and wherein said low dose radiation on neoplastic tissues causes anchors to form in the blood vessels within said neoplastic tissues that aids in latching of antibodies to anchors, allowing the antibodies to enter nearby neoplastic cells and kill them;
(b) waiting for a period of 48 to 72 hours and infusing a chemotherapeutic drug to act upon said neoplastic tissues.

2. The method of claim 1, wherein irradiating the non-neoplastic cells modulates one or more genetic pathway responsible for cell repair proteins.

3. The method of claim 2, wherein the modulation of genetic pathway by low dose radiation on the non-neoplastic cell can be used to determine the reaction of one or more pharmaceuticals or chemical agents on said one or more non-neoplastic cell.

4. The method of claim 2, wherein the modulation of genetic pathway by low dose radiation on the non-neoplastic cell can be used to protect against radiation hazards (e.g. radiation workers, first responders, and astronauts).

5. The method of claim 1, wherein the low dose radiation is in range of 5 cGy to 20 cGy.

6. The method of claim 1, wherein the non-neoplastic cells are in contact with or in close proximity to a target neoplastic cell of a neoplastic disease.

7. The method of claim 1, which is a method of therapeutic treatment of cancer with chemotherapy.

8. The method of claim 1, wherein the low dose can be administered by a neutron beam as well as a standard x-ray/gamma beam.

9. A method for killing cancerous cells comprising:
(a) targeting a tumor tissue, tissues sensitive to a chemo drug and one or more non-neoplastic cells present in vicinity of a tumor tissue with a predetermined low dose of radiation, wherein said low dose radiation induces an immune response against tumor tissues, a cellular repair process in said one or more non-neoplastic cell and tissues sensitive to the chemo drug; wherein the low dose radiation on the tumor tissue causes anchors to form in the blood vessels within said tumor tissue that aids in latching of antibodies to anchors, allowing the antibodies to enter nearby tumor cells and kill them;
(b) waiting for a period of 48 to 72 hours and infusing a chemotherapeutic drug to act upon the tumor tissue; and wherein the above steps are repeated till the recommended dose of high radiation is completed.

10. The method of claim 9, wherein low dose of radiation modulates one or more genetic pathway of non-neoplastic cells to induce cell repair proteins.

11. The method of claim 9, wherein the low dose radiation is in range of 5 cGy to 20 cGy.

12. The method of claim 9, wherein the immune response initiated by the low dose radiation inhibits the proliferation of neoplastic cells.

13. The method of claim 9, wherein the immune response protects the healthy non-neoplastic cells and tissues sensitive to the chemo drug from the chemo drug.

14. A method of killing cancerous cells comprising:
(a) administering a low dose radiation to neoplastic tissues, non-neoplastic cells surrounding neoplastic tissues and non-neoplastic cells sensitive to a chemo-drug;
wherein said low dose radiation elicits antibodies against neoplastic tissues and elicits a repair mechanism in the non-neoplastic cells and in the non-neoplastic cells sensitive to the chemo-drug;
and wherein said low dose radiation on neoplastic tissues causes anchors to form in the blood vessels within said neoplastic tissues that aids in latching of antibodies to anchors, allowing the antibodies to enter nearby neoplastic cells and kill them;
(b) waiting for a period of 48 to 72 hours and administering a second predetermined low dose radiation to the non-neoplastic cells surrounding neoplastic tissues and non-neoplastic cells sensitive to the chemo-drug, wherein said second predetermined low dose radiation elicit repair mechanism in said non-neoplastic cells;
(c) waiting for 24 hours and infusing a chemotherapeutic drug to act upon said neoplastic tissues.

15. The method of claim 14, wherein the low dose radiation is in range of 5 cGy to 15 cGy.

16. The method of claim 14, wherein the low dose radiation modulates the genes responsible for repair mechanism in the non-neoplastic cells.

17. The method of claim 14, wherein antibodies against neoplastic tissues prevent invading neoplastic cells from entering into non-neoplastic cells.

18. The method of claim 14, wherein the repair mechanism in non-neoplastic cells protects the cells from the chemo drugs.
